(19) United States
(12) Patent Application Publication
(10) Pub. No.: US 2023/0110676 A1
(43) Pub. Date: Apr. 13, 2023

(54) Method of Treating Diseases

(71) Applicant: Ergo-Health LLC, Aubumdale, MA (US)

(72) Inventor: Pinghua Liu, Newton, MA (US)

(73) Assignee: Ergo-Health LLC, Aubumdale, MA (US)

(21) Appl. No.: 17/904,977

(22) PCT Filed: Feb. 25, 2021
(86) PCT No.: PCT/US21/19620

§ 371 Date: Aug. 25, 2022

Related U.S. Application Data
(60) Provisional application No. 62/981,204, filed on Feb. 25, 2020.

(51) Int. Cl. A61K 31/4172; A61K 45/06; C12N 5/074; C12N 5/0735

(52) U.S. Cl. CPC A61K 31/4172; A61K 45/06; C12N 5/0696; C12N 5/0606; C12N 2501/999

(57) ABSTRACT

A method of treating diseases with the compound of Formula I is disclosed. The compound exhibits therapeutic effect to the treatment of various diseases including neurodegenerative diseases, muscular dystrophy, and cardiovascular diseases.

### CROSS-REFERENCE TO RELATED APPLICATION

This application claims priority under 35 U.S.C. § 119(e) to U.S. provisional Patent Application No. 62/981,204, filed Feb. 25, 2020, which is hereby incorporated by reference in its entirety.

### TECHNICAL FIELD

This patent document relates to a method of treating diseases including neurodegenerative diseases, muscular dystrophy, and cardiovascular diseases.

### BACKGROUND

Despite significant advances in therapy, neurodegenerative and cardiovascular disease remains a common cause of morbidity and mortality in the developed world. Thus, prevention and therapy of neurodegenerative and cardiovascular conditions such as Parkinson's disease, myocardial infarction and stroke is an area of major public health importance. Currently, several risk factors for future neurodegenerative and cardiovascular disorders have been described and are in wide clinical use in the detection of individuals at high risk. However, an urgent need still exists for effective treatment for these diseases.

### SUMMARY OF THE INVENTION

An aspect of this patent discloses a method of treating a subject suffering from or susceptible to a disease. The disease can be a neurodegenerative disease, muscular dystrophy, or cardiovascular disease. The method includes administering to the subject a therapeutically effective amount of a compound of Formula I (compound I), or a pharmaceutically acceptable salt thereof,

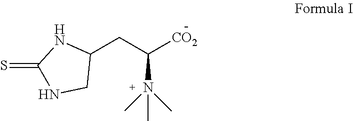

In some embodiments, at least 1, at least 3, or at least 5 mg of the compound, or the pharmaceutically acceptable salt thereof is administer per day to the subject. In some embodiments, the compound or pharmaceutically acceptable salt thereof is administered continuously at a predetermined daily dosage for a period of at least 5, at least 10, at least 14, at least 30, at least 60, or at least 180 days.

In some embodiments, the cardiovascular disease is selected from the group consisting of arrhythmia, ischemic heart disease, hypertensive heart disease and pulmonary hypertensive heart disease, valvular disease, and congenital heart disease. In some embodiments, the disease is a cardiovascular disease selected from myocardial infarction, cardiac hypertrophy, and arrhythmia. In some embodiments, the subject is diagnosed to have the cardiovascular disease, and the compound reverses or reduces remodeling resulting from the cardiovascular disease in the subject. In some embodiments, the subject is at risk to develop cardiovascular disease and the compound prevents disease initiation or progression. In some embodiments, the subject has suffered a previous cardiovascular disease event (e.g., myocardial infarction) and the compound reverses damage from the event or reduces the likelihood of a following adverse event.

The method may further include a step of determining that the subject is suffering from or susceptible to the cardiovascular disease. In some embodiments, the stage or progress of the cardiovascular disease is diagnosed and the dosage of the active ingredient is adjusted accordingly.

In some embodiments, the method further includes administering to the subject a secondary agent selected from the group consisting of antihyperlipoproteinemic, antiarteriosclerotic, antithrombotic, blood coagulant, antiarrhythmic agent, antihypertensive agent, vasopressor, diuretic, and inotropic agent.

In some embodiments, the disease is cardiovascular disease and the compound improves cardiac function by inducing cardiomyocyte proliferation. In some embodiments, the cardiomyocyte proliferation is sufficient to increase cardiac contractile force, or increase the thickness of the myocardium. In some embodiments, the method includes detecting an improvement in cardiac function, an increase in cardiac contractile force, or an increase in the thickness of the myocardium, and adjusting dosage of the compound accordingly. In some embodiments, the disease is cardiovascular disease and the compound functions by reducing or eliminating arrhythmia.

In some embodiments, the disease is selected from the group consisting of Alzheimer's disease, multiple sclerosis, Parkinson's disease, amyotrophic lateral sclerosis, cerebral ischemic disease, Huntington's disease, spinal muscular atrophy, stroke, brain trauma, spinal cord injury, prion disease and diabetic neuropathy. In some embodiments, the disease is selected from the group consisting of Alzheimer's disease, multiple sclerosis, Parkinson's disease, spinal muscular atrophy, Duchenne type muscular dystrophy, prion disease and stroke. In some embodiments, the method further includes determining the progress of the disease and adjusting the dosage of the compound accordingly.

Another aspect of the document provides a method of enhancing differentiation efficiency of a stem cell comprising contacting the stem cell with an effective amount of the compound of Formula I. In some embodiments, the stem cell is a totipotent, pluripotent, multipotent, oligopotent or unipotent stem cell. In some embodiments, the stem cell is a human embryonic stem cell or a human induced pluripotent stem cell.

In some embodiments, the stem cell develops into myocardiocytes or cardiac myocytes. In some embodiments, the stem cell develops into neural cells. In some embodiments, the stem cell develops into endothelial cells or immune cells (e.g., macrophage).

### BRIEF DESCRIPTION OF THE DRAWINGS

FIGS. 1A, 1B, 1C, and 1D show the life-extension effect of ergothioneine.

FIGS. 2A, 2B, and 2C show the effect of ergothioneine in daf-16, akt-2, and skn-1 mutants, respectively.

FIG. 3 shows the accelerated aging of mouse model.

FIGS. 4A and 4B show the animal study in rats. A. untreated disease group; B treated disease group.

FIGS. 5A and 5B show the physical activities of untreated disease group and treated disease group at week #57 (open field experiment). A) Real time trace of mouse movement. The left panel is the control group where most of the mice stay in the corners while the ergo-treated group (right panel) demonstrated much more activities. B) Quantitative measurement of the distance each mouse moved during the testing period and the time they spent in the open area.

FIG. 6 shows echocardiographic assessment of hearts from mice 4 weeks after MI and treated with EGT2. (*P<0.05, vs. Sham, ^{#}P<0.05, vs. MI, n=6 per group)

FIG. 7 shows ratio of heart weight to body weight in newly born mice (7 days). (*P<0.05, vs. CON, n=6×6)

FIG. 8 shows cardiomyocyte proliferation of NRVM (7 days). (*P<0.05, vs. CON, n=6×6).

FIG. 9 shows cardiomyocyte proliferation of NRVM (7 days). (*P<0.05, vs. CON, n=6×6).

FIG. 10 shows compound I's Beneficial Effect on Mouse Parkinson's Disease Model. Parkinson mouse model was induced by 1-methyl-4-phenyl-1,2,3,6-tetrahydropyridine (MPTP), which induced toxicity to dopaminergic neurons (latency test). Behavior test was done by testing mouse behavior: how long can they hold on rotatory plate/rod. The longer they stay, the better their health conditions. After MPTP treatment, mice are clearly much weaker. However, after feeding ergothioneine for 7 days, mice are clearly protected from these detrimental effects. Significance of difference was determined by unpaired two-tailed t-test. #P<0.05 compared with control, *P<0.05, **P<0.01, ****P<0.0001 compared with MPPP-treated control. 1) Latency test results of control group with no MPTP or ergothioneine; 2) Latency test results of mice treated with 20 mg/Kg of MPTP for seven days; 3) Latency test results of mice treated with 20 mg/Kg of MPTP and 10 mg/kg of ergothioneine (co-injection) for seven days; 4) Latency test results of mice treated with 20 mg/Kg of MPTP and 20 mg/kg of ergothioneine (co-injection) for seven days; 5) Latency test results of mice treated with 20 mg/Kg of MPTP and 40 mg/kg of ergothioneine in water for seven days. This result clearly shows the protective effect of ergothioneine in MPTP Parkinson's disease mouse model.

FIG. 11 shows compound I's Beneficial Effect on Mouse Parkinson's Disease Model. Parkinson mouse model was induced by MPTP. Behavior test was done by testing mouse behavior: how long does it take for the mice to climb to the top of a rod. The shorter time they took, the better their health conditions are. After MPTP treatment, mice are clearly much weaker. However, after feeding ergothioneine for 7 days, mice are clearly protected from these detrimental effects. Significance of difference was determined by unpaired two-tailed t-test. ###P<0.05 compared with control, ***P<0.001, ****P<0.0001 compared with MPPP-treated control. 1) Latency test results of control group with no MPTP or ergothioneine; 2) Latency test results of mice treated with 20 mg/Kg of MPTP for seven days; 3) Latency test results of mice treated with 20 mg/Kg of MPTP and 10 mg/kg of ergothioneine (co-injection) for seven days; 4) Latency test results of mice treated with 20 mg/Kg of MPTP and 20 mg/kg of ergothioneine (co-injection) for seven days; 5) Latency test results of mice treated with 20 mg/Kg of MPTP and 40 mg/kg of ergothioneine in water for seven days. The pole test result also clearly shows the protective effect of ergothioneine in MPTP Parkinson's disease mouse model.

FIG. 12 shows Ergothioneine's Protective Effect on MPP's Detrimental Effect (Preventive effect for Parkinson's Diseases). MPP+treatment significantly decrease worm's survival rate. However, ergothioneine can offer significant protection. The last column is the worm with no ergothioneine or MPTP treatment. The first column is the worm survival rate after treating with 0.75 mM of MPTP. When ergothioneine is also included (3.12-400 μM), worm's survival significantly increased from ˜50% (column 1) to nearly 90% with 400 μM of ergothioneine (column 2).

FIGS. 13A and B show Protective Effect for Heat-shock for Both Wild-type and a-Synuclein Overexpression Worm Model (Parkinson's disease). Heat shock experiment. α-synuclein was expressed to create worm parkinson's disease model on worm. In this disease model, ergothioneine significantly improve worm's tolerance to heat shock. 50% worm died in 3 hours while the presence of ergothioneine clearly improved the stress resistance capability. With 1.6 mM of ergothioneine, 50% survival time increased to 8 hours.

FIG. 14 shows acute ischemic stroke model results using Photothrombotic Middle Cerebral Artery Occlusion method. A) model: A laser operating at 568 nm was used to irradiate the distal middle cerebral artery at a power of 20 mW for 4 minutes. The laser beam was focused with a 30-cm focal length convex lens (KPX 112, Newport Corporation) and positioned with a mirror onto the distal middle cerebral artery. The photosensitizing dye rose bengal (15 mg/mL in 0.9% saline) was administered intravenously at a body dose of 20 mg/kg over 90 seconds starting simultaneously with 4 minutes of laser irradiation to induce occlusion; B) The infarct size was then measured after staining with. Left column is the control without any drug treatment. The middle one with 40 mg/Kg of ergothioneine treatment and the right one is the one with 10 mg/kg of Edaravone (EDV, an approved drug in Japan and South Korea).

FIG. 15 shows acute ischemic stroke model results using Photothrombotic Middle Cerebral Artery Occlusion method (picture of TTC staining result and quantitative analysis of infarct volume ratio). A) model: A laser operating at 568 nm was used to irradiate the distal middle cerebral artery at a power of 20 mW for 4 minutes. The laser beam was focused with a 30-cm focal length convex lens (KPX 112, Newport Corporation) and positioned with a mirror onto the distal middle cerebral artery. The photosensitizing dye rose bengal (15 mg/mL in 0.9% saline) was administered intravenously at a body dose of 20 mg/kg over 90 seconds starting simultaneously with 4 minutes of laser irradiation to induce occlusion; B) The infarct size was then measured after staining with. Left column is the control without any drug treatment. The middle one with 40 mg/Kg of ergothioneine treatment and the right one is the one with 10 mg/kg of Edaravone (EDV, an approved drug in Japan and South Korea).

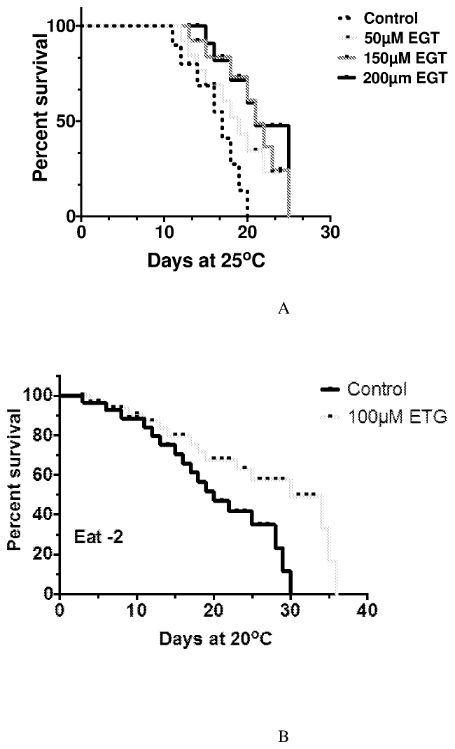

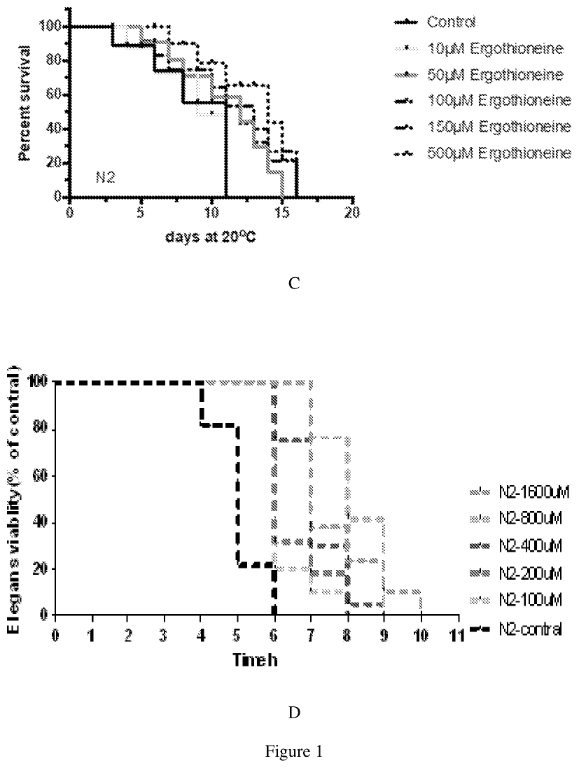

### DETAILED DESCRIPTION

The disclosed methods for treating a subject suffering from or susceptible to a disease are based on an unexpected discovery that the compound of Formula I have life-extension effect on a worm model under regular culture conditions and under stresses (e.g., oxidative stress conditions, heat-shock conditions). This disclosure further demonstrates that the compound exerts its life-extension effect through the daf-16 related signaling pathway. The discovery of ergothioneine's life-extension effect and the demonstration of the involvement of daf-16 opens the door for more systematic protective application on various chronic disease animal models. Results from animal studies have confirmed the therapeutic effect of the compound in diseases including neurological diseases, muscular diseases, and cardiovascular diseases.

While the following text may reference or exemplify specific embodiments of a method of treating a disease, it is not intended to limit the scope of the method to such particular reference or examples. Various modifications may be made by those skilled in the art, in view of practical and economic considerations, such as the dosage and administration of the compound for treatment of a particular disease.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” as used herein generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items. Expressions such as “at least one of,” when preceding a list of elements, modify the entire list of elements and do not modify the individual elements of the list.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder. The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder. “Treatment” also includes prophylactic treatment to prevent progression of the disease or for precautionary purpose for persons at risk of developing the disease or condition.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “diluent” refers to chemical compounds diluted in water that will dissolve the composition of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. As used herein, an “excipient” refers to an inert substance that is added to a composition to provide, without limitation, bulk, consistency, stability, binding ability, lubrication, disintegrating ability, etc., to the composition. A “diluent” is a type of excipient.

The term “physiologically acceptable” or “pharmaceutically acceptable” refers to a carrier or diluent that does not abrogate the biological activity and properties of the compound.

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The methods disclosed herein are based on the finding that compound I impacts FOXO signaling pathway, which is involved in responses to oxidative stress in mammalian cells and lifespan control in nematodes. The FOXO transcription factors influence cell metabolism, differentiation, and transformation. Meanwhile, misregulation of FOXO signaling contribute to the pathogenesis of various diseases and disorders.

DAF-16 is conserved across species, with homologs being found in C. elegans, humans, mice, and Drosophila (fruit flies). In C. elegans, DAF-16 is located on Chromosome 1, at position 175-268. It is made up of 15 exons. DAF-16 is also located downstream of DAF-2, which signals in the IIS pathway. Mutants in this pathway age slower and have a lifespan up to twice as long as normal. Further studies have demonstrated that the lifespan extension is dependent on DAF-16. Other consequences of mutations in the DAF-16 gene is the inability to form dauers.

DAF-16 encodes FOXO (Forkhead box protein O), which binds to gene promoters that contain the sequence TTGTTTAC in their regulatory region—this is the DAF-16 binding element (DBE). FOXO is involved in the Insulin/IGF1 signaling pathway (IIS) which affects longevity, lipogenesis, dauer formation, heat shock, and oxidative stress responses, by activating proteins such as MnSOD and Catalase. Expression of FOXO in the intestine normally leads to longevity signaling. FOXO has been shown to have a protective role against cancer, as it regulates and suppresses genes involved in tumor formation. It also has a protective role against muscular dystrophy. FOXO is also important in embryonic development, as it promotes apoptosis.

It has been discovered that Compound I can regulate the activities of Daf-16 and modify subsequent physiological processes. As is further demonstrated in the examples, Compound 1 exhibits potent activities in the enhancing cell viability, prolonging longevity, and improving mobility. Accordingly, the compound can be applied to the treatment and prevention of various diseases by regulating Daf-16 signal pathway.

An aspect of the patent document provides a method of treating a subject suffering from or susceptible to a disease by administering to a subject in need a therapeutically effective amount of Compound I or a pharmaceutical composition thereof. Through regulating the Daf-16 signal pathway, various diseases including neurologic/neurodegenerative diseases, neuromuscular diseases, muscular dystrophy, and cardiovascular diseases can be treated and/or prevented.

Neurologic or neurodegenerative disease as used herein refers to central nervous system disorders characterized by gradual and progressive loss of neural tissue and/or neural tissue function, with typically reduced neurological function as a result of a gradual and progressive loss of neural tissue. In some embodiments, the neurodegenerative diseases amenable to prevention and/or treatment using the methods as described herein are neurodegenerative diseases associated with aging or senescence in an individual. Non-limiting examples include Alzheimer's disease, multiple sclerosis, Parkinson's disease, amyotrophic lateral sclerosis, cerebral ischemic disease, Huntington's disease, spinal muscular atrophy, stroke, brain trauma, spinal cord injury, and diabetic neuropathy. Additional disorders that may be treated or prevented according to the methods described herein include those that involved oxidative stress, such as myocardial ischemic and peripheral ischemic disease; diabetic retinopathy, and diabetic nephropathy. In some embodiments, the method includes treating and/or preventing a disease selected from Alzheimer's disease, multiple sclerosis, Parkinson's disease, spinal muscular atrophy, prion disease and stroke,

Neuromuscular disease is often associated with neurodegenerative disease and is a broad term that encompasses many diseases and ailments that impair the functioning of the muscles, either directly, being pathologies of the voluntary muscle, or indirectly, being pathologies of nerves or neuromuscular junctions. Problems with central nervous control can cause either spasticity or some degree of paralysis (from both lower and upper motor neuron disorders), depending on the location and the nature of the problem. Some examples of central disorders include cerebrovascular accident, Parkinson's disease, multiple sclerosis, Huntington's disease, prion disease and Creutzfeldt-Jakob disease. Spinal muscular atrophies are disorders of lower motor neuron while amyotrophic lateral sclerosis is a mixed upper and lower motor neuron condition. In some embodiments, the method includes treating and/or preventing a disease selected from Parkinson's disease and spinal muscular atrophy.

In some embodiments, the disease to be treated is stroke. Stroke including ischemic stroke and a hemorrhagic stroke, along with heart disease and cancer, are the three leading cause of death and one of the most common diseases that affects life expectancy and prolonging the population's age as the frequency increases gradually with age. Hypertension, hyperlipidemia, atherosclerosis, and hypercholesterolemia are known as various important risk factors for cerebrovascular stroke.

Muscle atrophy leads to muscle weakness and causes disability. Muscle atrophy may or may not have a neurological cause. Muscle atrophy can be caused by various factors, including genetics, immobility, aging, malnutrition, medications, or a wide range of injuries or diseases that impact the musculoskeletal or nervous system. Non-limiting examples include spinal muscular atrophy, Duchenne type muscular dystrophy, and Becker muscular dystrophy. It also includes myotonic, facioscapulohumeral (fshd), congenital, limb-girdle muscle atrophy.

Cardiovascular disease or disorder or condition is intended to include all disorders characterized by insufficient, undesired or abnormal cardiac function, e.g., arrhythmia, ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The method described herein may also induce a reversal or reduction of the remodeling that occurs in hypertrophic or failing heart tissue in vivo, comprising administering to a subject suffering from cardiac hypertrophy or heart failure an effective amount of compound I that is sufficient to induce reverse remodeling, remodeling being defined as a decrease in the expression of the fetal genes and an increase in the expression of normal cardiac genes. Remodeling is a process to reverse the structural changes that occur in the heart in response to hypertrophy and heart failure. Secondary agents such as acetylcholine-esterase inhibitors and beta.-adrenergic receptor blockers to improve cardiac contractility and enhance systolic function of the heart.

Any of the methods described herein may include determining or diagnosing a subject as suffering from or being susceptible to the above described diseases, disorders or conditions. For instance, the method can include as step of identifying a subject as having a progressing myocardial infarction, stroke, or heart failure, or having a risk of developing myocardial infarction, stroke, or heart failure before administering the compound. The administration of compound I could effectively reduce such risk and prevent the disease or disorder from happening. Various procedures are known to diagnose a cardiovascular disease or disorder or determine the risk of having the disease or disorder. Exemplary procedures include those disclosed in U.S. Pat. Nos. 10,100,363, 10,352,947, U.S. Patent Application No. 20110086348 and 20090306181, and the references cited therein, the entire disclosure of which are incorporated by reference herein. Methods of determining the risk of neurologic/neurodegenerative diseases, neuromuscular diseases, or muscular dystrophy, or diagnosing these diseases or disorders are also known in the art. Exemplary procedures include those disclosed in U.S. Pat. Nos. 10,107,796, 10,085,688, 9,872,647, 9,820,671, 9,259,164, 8,577,451, and references disclosed therein, the entire disclosure of which are incorporated by reference herein.

In some embodiments, the method includes a step of determining the risk or progress of the above described disease, and adjusting the dosage of the compound accordingly. This step may be performed prior to the initial administration of the compound or at any interval during a period of administration. For instance, the initial daily dosage of the compound may be at least 1, at least 2, at least 5, at least 10, at least 20, at least 50, at least 100, at least 200, at least 500, at least 1000, at least 2000, or more than 3000 mg. After 1, 2, 3, 4, 8, 10, 20 or 40 weeks on such dosage, the subject may be examined to determine the effect of the compound and increase or reduce the dosage by 10%, 20%, 50%, 100%, or more than 200% depending on the effect of the compound.

Combinations

In some embodiments, the methods described herein includes administering a secondary agent which includes, without limitation, so-called “beta blockers,” anti-hypertensives, cardiotonics, anti-thrombotic s, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Additional examples of a secondary agent that may be used include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. In addition, it should be noted that any of the following may be used to develop new sets of cardiac therapy target genes. While it is expected that many of these genes may overlap, new gene targets likely can be developed. Further examples of secondary agents are disclosed in U.S. Pat. Nos. 7,485,653 and 9,737,569, the entire disclosure of which are incorporated by reference herein.

In some embodiments, the methods described herein includes administering a growth factor as a secondary agent. Suitable growth factors include, without limitation, a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNA-alpha), and placental growth factor (PLGF).

In some embodiments, the methods described herein includes administering a statin, i.e. a HMG-CoA reductase inhibitor as a secondary agent. Suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Additional examples of a secondary agent especially for neurodegenerative diseases (e.g. Parkinson's disease) include Levodopa, COMT inhibitors (e.g. Tolcapone, entacapone), MAO-B inhibitors (safinamide, selegiline and rasagiline), dopamine receptor agonists (e.g. ergoline and non ergoline agonists). Dopamine receptor agonists (sometimes also referred to as dopamine agonists) are substances which, while structurally different from dopamine, bind to different subtypes of dopamine receptors and trigger an effect which is comparable to that of dopamine. Non-limiting examples of dopamine receptor agonists include Bromocriptine, Pergolide, Pramipexole, Ropinirole, and Rotigotine. Additional examples include amantadine and anticholinergics.

Combinations may be achieved by contacting cardiac cells or administering to a subject with a single composition or pharmacological formulation that includes compound I and a secondary agent, or by contacting the cell or administering to a subject with two distinct compositions or formulations, at the same time. Alternatively, the therapy using the claimed formulations may precede or follow administration of the other agent(s) by intervals ranging from min to weeks. In embodiments where the various agents are applied separately to the cell, one would generally ensure that a significant period of time did not expire between the time of each delivery, such that the agents would still be able to exert an advantageously combined effect on the cell. In such instances, it is contemplated that one would typically contact the cell or administering to a subject with both modalities within about 12-24 hrs of each other and, more preferably, within about 6-12 hrs of each other, with a delay time of only about 12 hrs being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the claimed compounds, or the other agent will be desired. In this regard, various combinations may be employed. By way of illustration, where compound I is “A” and the other agent is “B,” the following permutations based on 3 and 4 total administrations are exemplary:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are likewise contemplated. Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Another aspect of the patent document discloses a method of enhancing differentiation efficiency of a stem cell, comprising contacting the stem cell with an effective amount of the compound of Formula I. In some embodiments, the stem cell has differentiated to form mesoderm. Non-limiting examples of the stem cells include a totipotent, pluripotent, multipotent, oligopotent and unipotent stem cell. In some embodiments, the stem cell is a human embryonic stem cell or a human induced pluripotent stem cell. In some embodiments, the stem cell develops into myocardiocytes or cardiac myocytes. In some embodiments, the stem cell develops into neural cells. The stem cell may be in a living organism or in an in vitro condition. New cells developed in in vitro condition from a stem cell may be transferred to a living organism (e.g. animal or human) for treatment of a disease or disorder.

Also provided herein are kits including the compound of Formula I for treating a subject suffering from or susceptible to a disease described above or for enhancing differentiation efficiency of a stem cell. For instance, a kit may comprise compound I, or a pharmaceutically acceptable prodrug, a pharmaceutically active metabolite, a pharmaceutically acceptable salt thereof described herein, a second agent as described above and optionally devices for contacting cells with compound I and/or the secondary agent. Devices include syringes, stents and other devices for administering compound I and/or the secondary agent into a subject. Further, a kit may also contain components for measuring a factor, e.g., a protein or transcript level, e.g., in tissue samples. The kit may also include instructions for use.

Dosage and Administration

The compound described herein or a pharmaceutical composition thereof may be administered to the subject by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; as well as (d) administration topically; as deemed appropriate by those of skill in the art for bringing the active compound into contact with living tissue.

The therapeutically effective amount of the compound disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

In non-human animal studies, applications of potential products are commenced at higher dosage levels, with dosage being decreased until the desired effect is no longer achieved adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively, dosages may be based and calculated upon the surface area of the patient, as understood by those of skill in the art.

The exact formulation, route of administration and dosage for the pharmaceutical compositions can be chosen by the individual physician in view of the patient's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of about 0.1 mg to 5000 mg of the active ingredient, preferably from about 1 mg to about 1000 mg, from about 1 mg to about 500 mg, from about 1 mg to about 500 mg, from about 10 mg to about 500 mg, from about 10 mg to about 100 mg, from about 10 mg to about 50 mg, from about 1 mg to about 500 mg from about 20 mg to about 500 mg, from about 50 mg to about 500 mg, from about 50 mg to about 100 mg, from about 100 mg to about 300 mg, or from about 100 mg to about 200 mg. In other embodiments, an intravenous, subcutaneous, or intramuscular dose of the active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free acid. In some embodiments, the composition is administered 1 to 4 times per day. Alternatively the compositions may be administered by continuous intravenous infusion, preferably at a dose of up to about 2000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety, which are sufficient to maintain a minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the compound may not be related to plasma concentration.

The compound disclosed herein can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of the compound in humans.

The compound of compositions thereof may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

In some embodiments, in the pharmaceutical industry, it is standard practice to provide substantially pure material when formulating pharmaceutical compositions. Therefore, in some embodiments, “substantially pure” refers to the amount of purity required for formulating pharmaceuticals, which may include, for example, a small amount of other material that will not affect the suitability for pharmaceutical use. In some embodiments, the substantially pure compound contains at least about 96% of the compound by weight, such as at least about 97%, 98%, 99%, or 100% of the compound.

### EXAMPLE 1

In this example, the biological functions of ergothioneine were investigated using C. elegans as the model system. Using C. elegans as the model has several prominent advantages, including short life cycle, simple experimental manipulation, and rich collection of publicly available genetic mutants (e.g., Caenorhabditis Genetics Center). In addition, its genome comprises ⅔ of human disease-related genes.

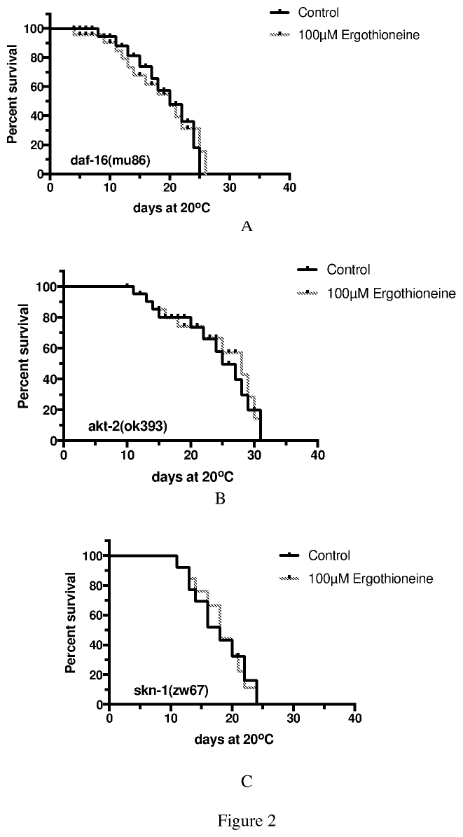

To test the biological effect of ergothioneine, the first set of experiments was to test the toxicity level of ergothioneine on the worm model. In humans, ergothioneine is enriched up to 2 mM concentration in many parts of the body. Surprisingly, even at concentrations as low as 50 μM, ergothioneine demonstrated a robust life-extension effect on N2 C. elegans (FIG. 2A). Encouraged by this result, ergothioneine concentration dependence was then examined. At 150 μM, the lifespan of the wild type worm can be extended by 30-40% (FIG. 2A). Over the last two decades, there is numerous evidence supporting the life-extension effect by caloric restriction from various model systems (yeast, worm, fly, and mouse). Thus, the effect of ergothioneine on worm Eat-2 mutant was also examined. Mutations in eat genes resulted in partial starvation of the worm by disrupting the function of the pharynx and significantly increase the lifespan. Interestingly, ergothioneine can further extend the life of Eat-2 worm mutant (FIG. 2B). This result suggests that ergothioneine can work synergistically with the currently known caloric restriction approach to further extend the lifespan (FIG. 2C).

### EXAMPLE 2

Encouraged by the clear life-extension effect on worm model under regular culture conditions in the above example, the protective effect of ergothioneine under stresses was examined. In the first experiment, the worm was treated with paraquat to induce oxidative stress. Under the paraquat-induced oxidative stress conditions, N2 worms' life span was decreased by nearly 50% (FIG. 2C vs. FIG. 2A). As anticipated, when ergothioneine was added to worms under oxidative stress conditions, it clearly offers protective effects and alleviates the detrimental effects of oxidative stress (FIG. 2C).

Besides the oxidative stress, the protective effect of ergothioneine for the worm under heat shock conditions was also examined. After the worms were cultured multiple days, they were then moved from 20° C. to 42° C. Under such a heat-shock condition, 50% of the worm died within 3 hours. However, in the presence of a few hundred micromolar concentration of ergothioneine, it extends N2 worm's average survival time from 3 hours to nearly 8-9 hours (FIG. 2D). Therefore, besides its life-extension effects under regular culture conditions, ergothioneine clearly offers protective effects to worms under oxidative stress and heat shock conditions.

### EXAMPLE 4

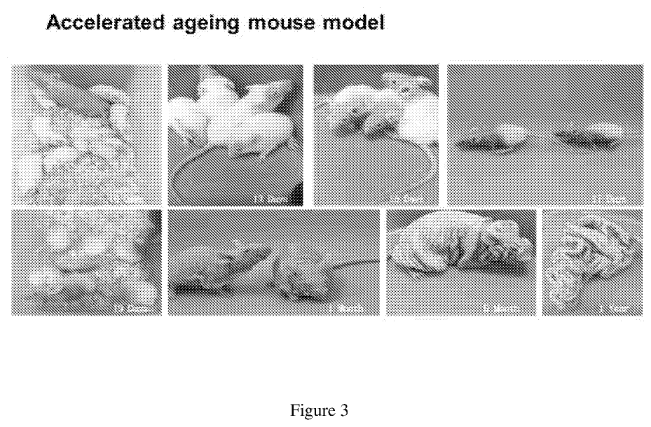

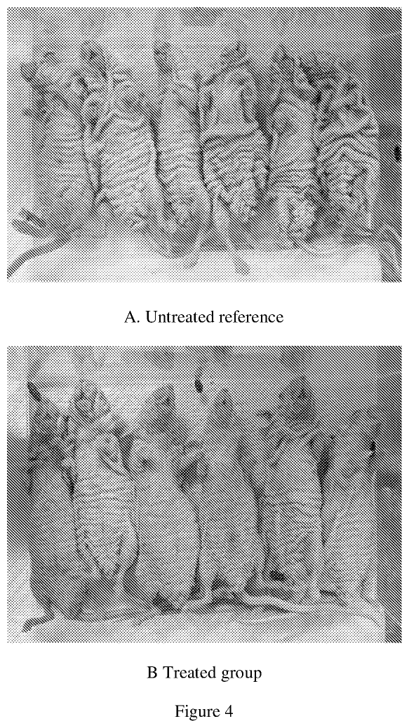

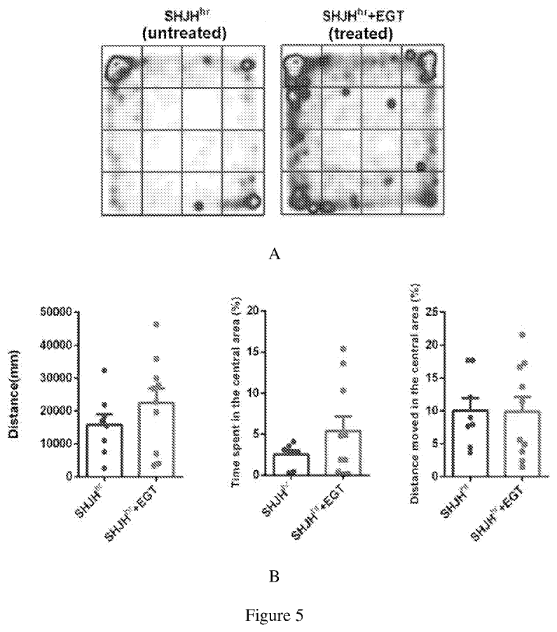

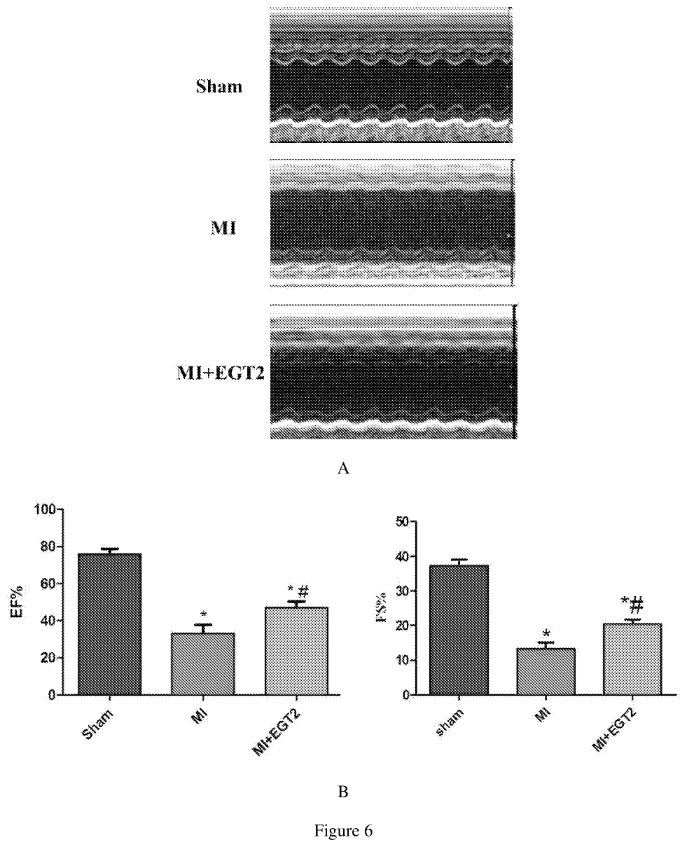

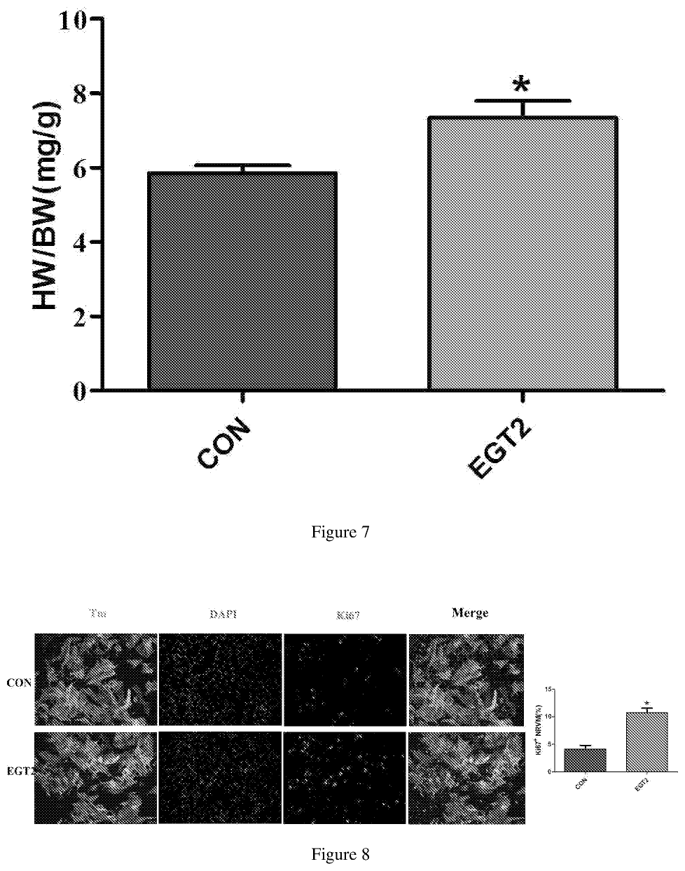

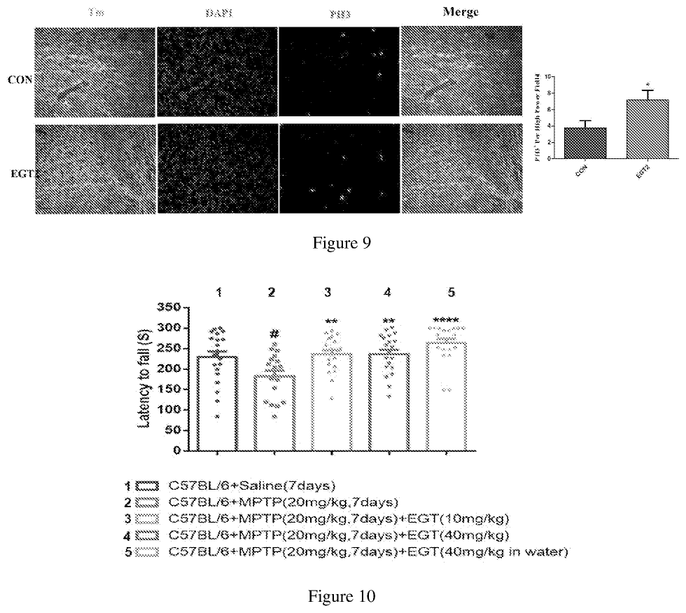

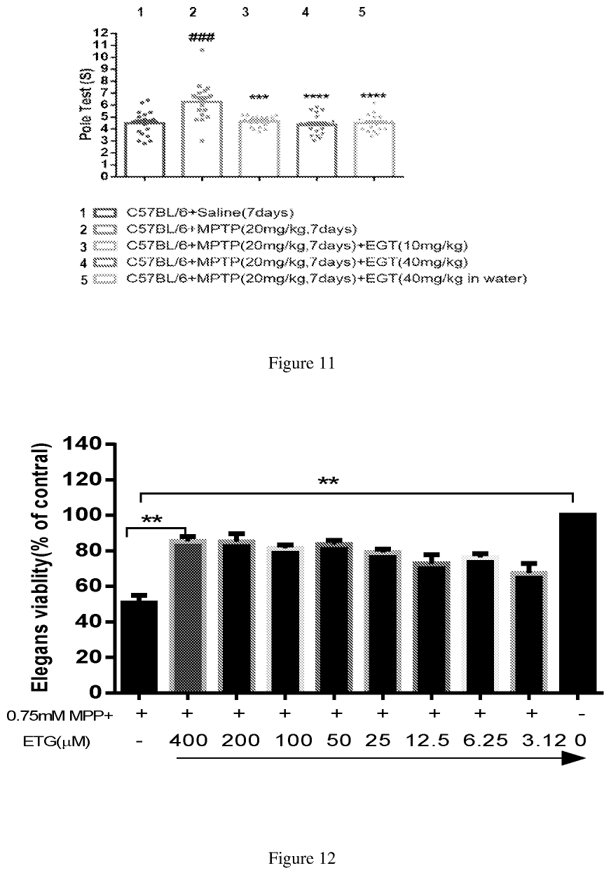

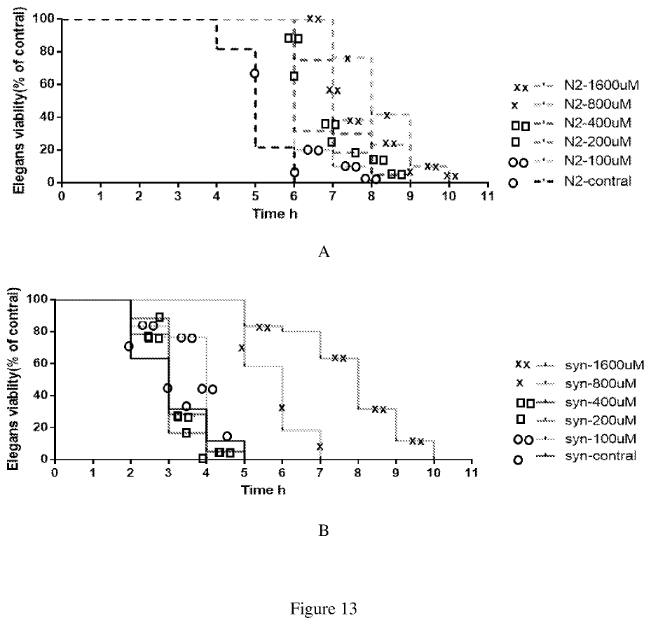

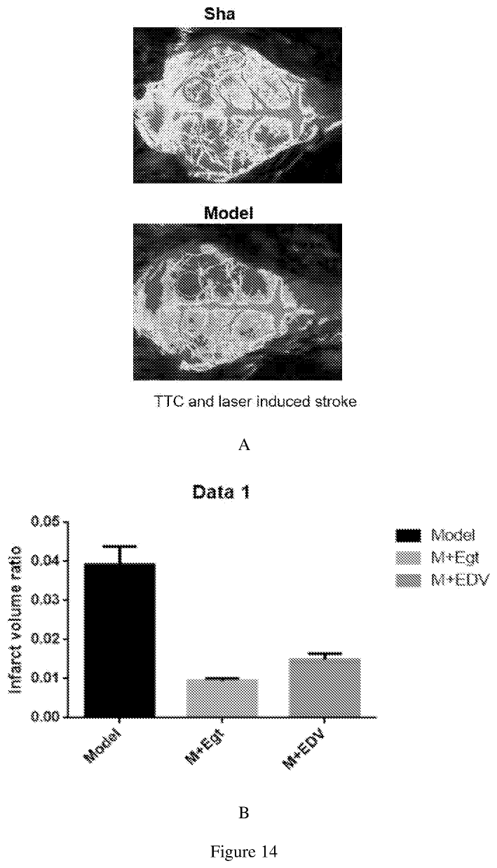

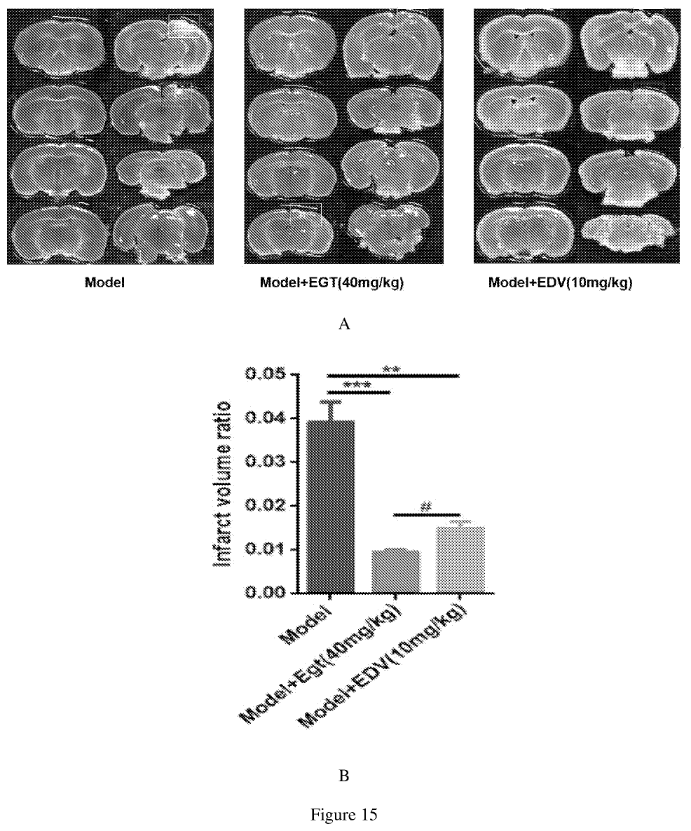

After demonstrating the life-extension effect of ergothioneine under both regular and stress conditions on the N2 worm model, it remained to be determined what signaling pathway might be involved. To address this issue, the large collection of C. elegans mutants in Caenorhabditis Genetics Center was again utilized in this example. Among the mutants examined, the daf-16 mutant results stood out (FIGS. 3A, 3B, and 3C). Insulin/IGF-1 signaling (IIS) pathway has been demonstrated to be related to worm life-span. Among genes of the IIS pathway, daf-2 encodes an insulin-like receptor, and it is the starting point of the pathway that eventually leads to daf-16. Daf-16 is the hub, where information from several different pathways merge and then use to regulate many physiological processes. Worm daf-16 mutant has an average life-span that is almost twice as long as the N2 worm (FIGS. 3A, 3B, and 3C). Interestingly, when ergothioneine was added onto the daf-16 mutant worm, the life-extension effect was abolished. After decades of searching for ergothioneine related working mechanism, for the first time, this result now clearly shows that ergothioneine most likely work through daf-16 related signaling pathways.

It will be appreciated by persons skilled in the art that methods described herein are not limited to what has been particularly shown and described. Rather, the scope of the method of treatment is defined by the claims which follow. It should further be understood that the above description is only representative of illustrative examples of embodiments. The description has not attempted to exhaustively enumerate all possible variations. The alternate embodiments may not have been presented for a specific step or component of the method, and may result from a different combination of described steps or components, or that other un-described alternate embodiments may be available for a step or component, is not to be considered a disclaimer of those alternate embodiments. It will be appreciated that many of those un-described embodiments are within the literal scope of the following claims, and others are equivalent.

1. A method of treating a subject suffering from or susceptible to a disease selected from the group consisting of neurodegenerative diseases, muscular dystrophy, and cardiovascular diseases, comprising administering to the subject a therapeutically effective amount of a compound of Formula I, or a pharmaceutically acceptable salt thereof,

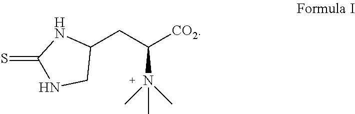

2. The method of claim 1, wherein at least about 5 mg of the compound, or the pharmaceutically acceptable salt thereof is administer a day to the subject.

3. The method of claim 1, wherein the compound or pharmaceutically acceptable salt thereof is administered continuously for at least a period of 30 days.

4. The method of claim 1, further comprising a step of determining that the subject is suffering from or susceptible to the disease.

5. The method of claim 1, wherein the subject is diagnosed to have the cardiovascular disease, and the compound reverses or reduces remodeling resulting from the cardiovascular disease in the subject.

6. The method of claim 1, further comprising administering to the subject a secondary agent selected from the group consisting of antihyperlipoproteinemic, antiarteriosclerotic, antithrombotic, blood coagulant, antiarrhythmic agent, antihypertensive agent, vasopressor, diuretic, and inotropic agent.

7. The method of claim 1, further comprising a step of determining that the subject is suffering from or susceptible to the cardiovascular disease.

8. The method of claim 1, wherein a stage or progress of the cardiovascular disease is diagnosed and the dosage of the active ingredient is adjusted accordingly.

9. The method of claim 1, wherein the disease is cardiovascular disease and the compound improves cardiac function by inducing cardiomyocyte proliferation.

10. The method of claim 9, wherein the cardiomyocyte proliferation is sufficient to increase cardiac contractile force or increase the thickness of the myocardium.

11. The method of claim 9, further comprising detecting an improvement in cardiac function, an increase in cardiac contractile force, or an increase in the thickness of the myocardium, and adjusting dosage of the compound accordingly.

12. The method of claim 1, wherein the cardiovascular disease is selected from the group consisting of arrhythmia, ischemic heart disease, hypertensive heart disease and pulmonary hypertensive heart disease, valvular disease, and congenital heart disease.

13. The method of claim 1, wherein the cardiovascular disease is myocardial infarction, cardiac hypertrophy, or arrhythmia.

14. The method of claim 1, wherein the disease is selected from the group consisting of Alzheimer's disease, multiple sclerosis, Parkinson's disease, amyotrophic lateral sclerosis, cerebral ischemic disease, Huntington's disease, spinal muscular atrophy, stroke, brain trauma, spinal cord injury, prion disease and diabetic neuropathy.

15. The method of claim 1, wherein the disease is selected from the group consisting of Alzheimer's disease, multiple sclerosis, Parkinson's disease, spinal muscular atrophy, Duchenne type muscular dystrophy, prion disease and stroke.

16. The method of claim 1, further comprising determining the progress of the disease and adjusting the dosage of the compound accordingly.

17. A method of enhancing differentiation efficiency of a stem cell, comprising contacting the stem cell with an effective amount of the compound of Formula I.

18. The method of claim 17, wherein the stem cell is a totipotent, pluripotent, multipotent, oligopotent or unipotent stem cell.

19. The method of claim 17, wherein the stem cell is a human embryonic stem cell or a human induced pluripotent stem cell.

20. The method of claim 17, wherein the stem cell develops into myocardiocytes or cardiac myocytes, endothelial cells or immune cells.

21. (canceled)
